(19) United States
(12) Patent Application Publication
(10) Pub. No.: US 2021/0315083 A1
(43) Pub. Date: Oct. 7, 2021

(54) CIRCADIAN OUTDOOR EQUIVALENCY METRIC FOR ASSESSING PHOTIC ENVIRONMENT AND HISTORY

(71) Applicant: ECOSENSE LIGHTING, INC., Los Angeles, CA (US)

(72) Inventors: Benjamin Harrison, LOS ANGELES, CA (US); Raghuram L.V. Petluri, LOS ANGELES, CA (US); Paul Kenneth Pickard, LOS ANGELES, CA (US)

(21) Appl. No.: 17/176,902

(22) Filed: Feb. 16, 2021

Related U.S. Application Data
(60) Provisional application No. 62/977,479, filed on Feb. 17, 2020.

(51) Int. Cl. H05B 47/11; H05B 47/16; H05B 45/22; G09G 5/12

(52) U.S. Cl. CPC H05B 47/11; H05B 47/16; G09G 2360/144; G09G 5/12; H05B 45/22

(57) ABSTRACT

A computer-implemented method, includes obtaining information about a photic environment, the information including at least one light metric, tracking the at least one metric over a period of time, generating a dosage level based on information about the photic environment, the tracked metric, and an intended circadian response, wherein the dosage level includes a light level and outputting the dosage level on at least one light-emitting device.

### CROSS REFERENCE TO RELATED APPLICATIONS

This application claims the benefit of U.S. Provisional Patent Appl. No. 62/977,479, filed Feb. 17, 2020 the entire disclosure of which is incorporated herein by reference.

### FIELD OF THE DISCLOSURE

This disclosure is in the field bioactive digital display devices. In particular, the disclosure relates to devices for use in, and methods of, obtaining and applying photic environment data to provide controllable biological effects from bioactive lighting systems, such as bioactive display systems.

### BACKGROUND

Circadian rhythms are found in virtually all organisms on earth. They are characterized by cyclical variations over a 24-hour period. They allow organisms to anticipate changes in their environment and to exploit them to their advantage. Many organisms contain one or more “clocks” that free run with a period close to 24 hours, but generally not exactly. These clocks entrain to the environment via external time cues, known as zeitgebers. There are many zeitgebers, but the most powerful is the 24-hour cycle of light and dark. Others include food intake, exercise, social interactions and temperature variation.

The human body is known to contain thousands of clocks, with more being discovered every year. These clocks are independent but are synchronized via a signal from the master clock, the suprachiasmatic nucleus (SCN). The SCN integrates external cues, or zeitgebers, to track the time of day. Light, a major zeitgeber, enters the eye and stimulates ipRGCs. This stimulation causes a signal to be transmitted to the SCN, which then knows that the organism is in a lit environment and it is therefore daytime. The strength of the zeitgeber influences the strength of the signal given to the body by the SCN. The strength of this signal determines the extent to which the individual clocks are correctly synchronized. A weak signal leads to poor synchrony which leads to dephasing of individual clocks which then leads to a decrease in health and wellness.

Prior to the invention of electric light, human exposure to light was driven by the sun. During the day, light levels are very high, typically between 1,000 lux and 100,000 lux. At night, with darkness, light levels are generally below 1 lux, with a full moon on a clear night providing 0.25 lux and a cloudy sky with a new moon providing 0.0001 lux. Today, individuals spend a great deal of time indoors, with the average individual spending 93% of their life inside. Indoor spaces are lit with electric light and can be 1000 times dimmer than the outside during the day and 1,000 times brighter at night. Further to this intensity change, timing of exposure to light is now governed by behavior and not by the sun. For example, a person may sleep in a darkened room and turn the lights on when they wake. Depending on the individual, this timing may vary considerably from day to day. This is contrary to the stable timing of the sunrise and can lead to circadian rhythm instability.

There are a wide variety of light emitting devices known in the art including, for example, incandescent light bulbs, fluorescent lights, and semiconductor light emitting devices such as light emitting diodes (“LEDs”), and many with variable light emitting levels. Accordingly, it may be beneficial to utilize the light levels such devices to help stabilize and/or otherwise impact circadian rhythm of organisms.

### SUMMARY

In some aspects, the present disclosure provides methods, systems and devices for providing a circadian outdoor equivalency metric for assessing photic environment and history. An outdoor equivalency metric may be applied to various lighting systems, networks, applications, and devices, for example, to influence one or more circadian responses. In various examples and embodiments, information about an environment, e.g., indoor or outdoor, and the lighting in the environment may be used to assess one or more lighting metrics. Such measurements and metrics may then be used to generate dosage levels, e.g., ideal dosage levels, to simulate a particular environment and/or to stimulate a particular circadian response. In some embodiments attributes describing these dosage levels may be communicated to an individual via a user interface. A plurality of lighting devices may generate a network of devices in one or more environments such that a user will be exposed to light levels that are intended or ideal for the user. As one example, the lighting network, comprised of a plurality of devices may simulate daytime or nighttime. In other embodiments, the devices may vary their light output depending on one or more factors such as a time of day and/or feedback from one or more metrics and information about the photic environment.

In an example, an embodiment may comprise systems, computer implemented methods, and devices that can obtain information about a photic environment, including at least one light metric; track the at least one metric over a period of time; generate a dosage level based on information about the photic environment, the tracked metric, and an intended circadian response, the dosage level including a light level; and output the dosage level on at least one light-emitting device.

In various embodiments, the at least one metric of the photic environment includes at least one of a daytime light intensity, a morning light intensity, an afternoon light intensity, an evening light intensity, a nighttime light intensity, a duration of night and day, a timing of dawn and dusk, a fraction of time spent in light intensity zone, a photopic lux, a melanopic lux, a circadian potency, a circadian light, and a scotopic lux.

In aspects of embodiments, obtaining information about the photic environment further comprises inferring, from non-light information, including at least one of a wearable device, a time, and a location. In some instances physiological sensors comprise one or more wearable devices incorporated in armbands, wrist bands, chest bands, glasses, or clothing. Aspects of the control methods include the physiological sensors configured to sense one or more of a person's temperature, blood pressure, heart rate, oxygen saturation, activity type, activity level, galvanic skin response, respiratory rate, cholesterol level (including HDL, LDL and triglyceride), hormone or adrenal levels (e.g., Cortisol, thyroid, adrenaline, melatonin, and others), histamine levels, immune system characteristics, blood alcohol levels, drug content, macro and micro nutrients, mood, emotional state, alertness, and sleepiness.

Dose response curves may be used when processing the light information or other information about the photic environment. In examples, the dose response curve can be based on a light level. In embodiments, the generated dosage level comprises an exposure time and/or a light level, based on one or more factors such as time, time of day, a phase angle between zeitgebers, information about one or more zeitgebers, an equivalent latitude, and equivalent longitude, and a risk factor. In addition, the intended circadian response of the dosage level may be any of a plurality of health and bioactive considerations and effects, such as improved sleep.

In various configurations of embodiments as disclosed herein, systems, networks and devices may include one or more processors and memories in a master device, e.g., a server, which may be local or remote to the photic environment and/or environments in which the one or more devices are located. Such light-emitting device may include but are not limited to a computer display, a LCD configuration, an OLED array, a uLED emissive display, a mobile device, a television display, a household, a SAD lamp, a lamp, a monitor, a blood pressure monitor, a wall panel, a light fixture, a multi-channel display system.

Aspects of various control systems and methods as discussed herein may comprise: a plurality of light emitting device outputting circadian stimulating energy (CSE); at least one external device receiving feedback comprising information associated with at least one of the semiconductor light emitting devices and the first CSE; and a master device in communication with the plurality of semiconductor light emitting devices, the master device configured to adjust a parameter on at least one of the plurality of semiconductor light emitting devices based on the feedback, and cause the at least one semiconductor light emitting devices to emit a second CSE.

In various examples, the external device may be a display system, wherein the display system comprises: one or more LED-based lighting channels adapted to generate a circadian-inducing blue light output in a first operational mode; a less circadian-inducing blue light output in a second operational mode; and a long red near infrared energy (LRNE) output in a third operating mode. In aspects of embodiments, the LRNE may be in at least one of the visible and the non-visible spectrum.

In additional aspects of control systems and methods, the one external device is a mobile device, a wearable device, a sensor, a panel system, a lighting device,

and a computing system. As discussed herein, the external device may be configured to sense one or more of temperature, pressure, ambient lighting conditions, localized lighting conditions, lighting spectrum characteristics, humidity, UV light, sound, particles, pollutants, gases, radiation, location of objects or items, and motion. In examples, the wearable device is incorporated in at least one of armbands, wrist bands, chest bands, glasses, or clothing.

In additional aspects, the one or more external devices are configured to sense one or more of a person's temperature, blood pressure, heart rate, oxygen saturation, activity type, activity level, galvanic skin response, respiratory rate, cholesterol level (including HDL, LDL and triglyceride), hormone or adrenal levels (e.g., Cortisol, thyroid, adrenaline, melatonin, and others), histamine levels, immune system characteristics, blood alcohol levels, drug content, macro and micro nutrients, mood, emotional state, alertness, and sleepiness. In examples, the feedback is indicative of information relating to at least one of light, motion, temperature, environment, physiological data, usage patterns, user feedback, and location.

a computer-implemented method, includes obtaining information about a photic environment, the information including at least one light metric, tracking the at least one metric over a period of time, generating a dosage level based on information about the photic environment, the tracked metric, and an intended circadian response, wherein the dosage level includes a light level and outputting the dosage level on at least one light-emitting device.

In accordance with an exemplary and non-limiting embodiment, a system comprises at least one light-emitting device, at least one processor in communication with the at least one light-emitting device and a memory in communication with the at least one processor, the memory comprising instructions executable by the at least one processor to at least obtain information about a photic environment, the information including at least one light metric, track the at least one metric over a period of time, generate a dosage level based on information about the photic environment, the tracked metric, and an intended circadian response, wherein the dosage level includes a light level and output the dosage level on at least one light-emitting device.

In accordance with an exemplary and non-limiting embodiment, a non-transitory computer-readable medium comprises instructions executable by at least one processor to perform a method, the method comprising obtaining information about a photic environment, the information including at least one light metric, tracking the at least one metric over a period of time, generating a dosage level based on information about the photic environment, the tracked metric, and an intended circadian response, wherein the dosage level includes a light level and outputting the dosage level on at least one light-emitting device.

In aspects, with respect to the master device, the master device may be at least one of a mobile device, a wearable device, and a computing device, and may be configured to receive user input. Additionally, the parameter may be associated with lighting control based on at least one of physiological factors, health conditions, emotional states, user mood, and user input. The master device may also be in communication with the plurality of semiconductor light emitting devices through one or more of a wired network, a wireless network, and Bluetooth communication.

The general disclosure and the following further disclosure are exemplary and explanatory only and are not restrictive of the disclosure, as defined in the appended claims. Other aspects of the present disclosure will be apparent to those skilled in the art in view of the details as provided herein. In the figures, like reference numerals designate corresponding parts throughout the different views. All callouts and annotations are hereby incorporated by this reference as if fully set forth herein.

### BRIEF DESCRIPTION OF THE DRAWINGS

The summary, as well as the following detailed description, is further understood when read in conjunction with the appended drawings. For the purpose of illustrating the disclosure, there are shown in the drawings exemplary implementations of the disclosure; however, the disclosure is not limited to the specific methods, compositions, and devices disclosed. In addition, the drawings are not necessarily drawn to scale. In the drawings:

FIG. 1 illustrates a flow chart for analyzing a photic environment in accordance with embodiments discussed herein;

FIG. 2 depicts aspects of a control system used in analyzing a photic environment, in accordance with embodiments discussed herein;

FIG. 3 is a block diagram of computing systems and methods usable with embodiments discussed herein; and,

FIG. 4 is an overview of a computing systems in accordance with embodiments discussed herein.

### DETAILED DESCRIPTION

The present disclosure may be understood more readily by reference to the following detailed description taken in connection with the accompanying figures and examples, which form a part of this disclosure. It is to be understood that this disclosure is not limited to the specific devices, methods, applications, conditions or parameters described and/or shown herein, and that the terminology used herein is for the purpose of describing particular exemplars by way of example only and is not intended to be limiting of the claimed disclosure. Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another exemplar includes from the one particular value and/or to the other particular value. Similarly, when values are expressed as approximations, by use of the antecedent “about,” it will be understood that the particular value forms another exemplar. All ranges are inclusive and combinable.

The term “circadian-stimulating energy characteristics” refers to any characteristics of a spectral power distribution that may have biological effects on a subject. In some aspects, the circadian-stimulating energy characteristics of aspects of the lighting systems of this disclosure can include one or more of CS, CLA, EML, BLH, CER, CAF, LEF, circadian power, circadian flux, and the relative amount of power within one or more particular wavelength ranges. Circadian-stimulating energy may be referred to as “CSE”. The application of CSE to biological systems in doses, amount, aliquots and volumes may be referred to as CSE therapy.

### Outdoor Equivalency

Outdoor Equivalency (OE) is predicated, in part, on the belief that humans evolved in the presence of a 24-hour cycle of light and dark and that negligible time, from an evolutionary perspective, has passed since the invention of electric light, and that, therefore, the natural cycle of light and dark as found in an outdoor environment constitutes an ideal reference signal for natural and healthy human circadian entrainment. OE draws from state-of-the-art understanding of how the body detects light, its sensitivity (i.e., dose-response) across intensity, wavelength and time and a myriad of other details. OE will track a number of aspects of the photic environment. These can then be combined, linearly or otherwise, to create one or more summary metrics. Over time as data is collected, such aspects, combinations and summaries will be refined at both the individual and population levels.

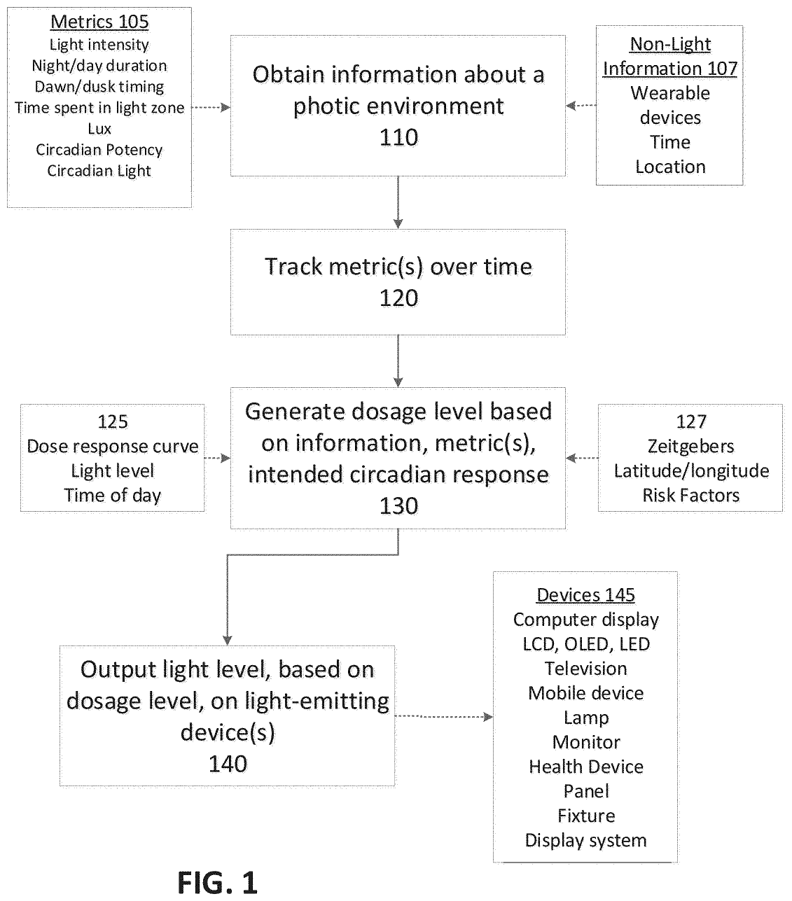

As illustrated in FIG. 1, which illustrates a flow chart demonstrating a method for analyzing a photic environment, information about an individual's photic environment 110 may be gathered in a number of ways. The information may be inferred from light information metrics 105 and non-light information 107. For example, a wearable device might detect that the wearer is walking outside. Knowing time and location, the system will be able to reasonably approximate the photic environment during the walk. In another example, the system may know that the individual is at home. The home may be equipped with smart lighting and the system may have access to the current settings and will therefore be able to approximate the individual's environment. Information may be gathered from existing wearable technologies, for example smart watches. This information will not perfectly match light entering the eye, but, with appropriate signal processing, may provide a good approximation. In the future, technology may be developed allowing for a more direct measurement of photic environment. Examples include, but are not limited to, contact lenses with the ability to measure and record spectral information, implants, jewelry, spectacles, or devices designed to be stuck directly to the face.

In addition to inferring the attributes of a photic environment, such attributes may also be directly measured. In some instances, a combination of inference and measuring of the photic environment may be utilized.

Key aspects of the photic environment may include light-related metrics 105, such as daytime light intensity, morning light intensity, afternoon light intensity, evening light intensity, nighttime light intensity, duration of night and day, timing of dawn and dusk, fraction of time spent in light intensity zone and others. Light intensity can be taken to mean any of photopic lux, melanopic lux, circadian potency, circadian light, scotopic lux or other.

For some aspects, it will be insightful to track responses across time 120. The pertinent timescale may range from days to a lifetime, depending on the application. One aspect where this is likely to be particularly important is dawn timing. The circadian system is particularly sensitive to light at sunrise. Variations in timing of exposure to first light will lead to variations in circadian phase. Stability in this metric will be a key indicator of overall circadian stability.

Dose response curves 125 may be used to process raw data. In other words, circadian responses from various light levels and stimuli may be collected to identify trends, patterns, and responses, as well as for processing purposes as further described herein. Such dose response curves may be sigmoidal in form or have other functional forms. One example of a dose response curve is the conversion of circadian light to circadian stimulus. In some embodiments, chronotypes may be considered when building a circadian response model for an individual.

Susceptibility to phase advances and delays is a function of time, so it is likely that an outdoor equivalency metric will weight doses differently throughout the day.

Photic environment data may be processed in any of a plurality of ways to calculate metadata for generating a dosage level 130. For example, information 127 such as the phase angle of the associated zeitgeber, e.g., a difference between the clock of the zeitgeber and another clock, may be processed to generate information about the photic environment. Other clocks may include the body clock of the individual, the local time zone and the time as determined by the sun. Other metadata may be calculated. For example, the individuals season based on photic environment, equivalent latitude and longitude, risk factor for seasonal affective disorder etc.

Once a dosage level is generated, based on light information, metric(s), and perhaps even an intended circadian response, one or more light-emitting devices may output a light level, based on the dosage level 140.

In accordance with some exemplary embodiments, the system may interpret measurable circadian cycle indicative conditions (MCCICs) in terms of an alignment of the MCCICs with some external time. In other embodiments, the system may further consider the relative alignment of one or more MCCICs in order to compute an optimum, or more optimal, alignment of more than one MCCIC tailored to one or more objectives, e.g., sleep, longevity, performance, etc.

It is anticipated that OE can be used in conjunction with data relating to other aspects of the human condition. This may be used to refine the algorithms used to calculate OE or to generate OE values for specific applications, or intended circadian responses, like increasing sleep quality, increased immunity, support of weight loss, improved mental health, improved fertility, improved athletic performance, improved academic performance and others.

It is also anticipated that OE will be used in closed-loop situations. For example, an individual may have a specific goal, such as improved sleep, and is therefore trying to achieve an OE above a certain threshold. This individual may be part of a greater ecosystem of light-emitting networks and devices 145. For example, a sunrise simulator, a mobile device equipped with display technology, a SAD lamp, residential lamps, a task lamp at work, computer monitors, a TV, a two-tone blood pressure monitor, a makeup mirror and so on. The OE system will inform a control system about the individual's progress towards their goal. The control system can then modify the individual's photic environment via one or more of their devices. Further layers of closed-loopedness may exist, for example with adjustments being made to the target OE value based on feedback from the individual's sleep patterns.

In accordance with exemplary embodiments, the generation of a dosage level and outputting of a light level may extend beyond a one to one mapping. For example, office lights impact everyone in the office, a TV at home influences the whole family, etc. Given each individual has their own needs, there is some degree of competition over the correct settings for a device impacting multiple people. The system may operate to partially or fully optimize performance to meet these various needs. This decision may include dose and phase response curves. People in more critical periods of their phase response may be prioritized, as would those further from where they need to be. In yet other exemplary embodiments, a person may be influenced by multiple devices at a time. Each of these devices may have some accessible range of outputs. A target effect may be distributed across the multiple devices taking their individual capabilities into account.

### Displays

Aspects of the present inventions relate to display systems that are adapted to produce and display color(s) at the pixel level that can be used to help in inducing and/or regulating a circadian rhythm cycle in a person looking at the displays or otherwise proximate the display. The display systems may be computer displays or television displays. The lighting system for the display systems pixels may be arranged to produce colors of the pixels in the display that effect the circadian rhythm over the course of time. The lighting system may be adapted to generate a circadian stimulating energy (CSE) blue frequency of light (e.g., cyan, energy at and/or near 485 nm) that causes activity associated with ‘waking’ the person through the circadian cycle (e.g., effecting, causing, or maintaining a wakeful and alert state in the viewer by enabling melatonin suppression by exciting the Intrinsically photosensitive retinal ganglion cells (ipRGCs)). It may also be adapted to reduce the circadian-inducing blue frequency over time to reduce the ‘waking’ effect. The lighting system may further be adapted with two or more separate blue frequencies such that either or both may be used to generate the blue in the pixels of the display. One of the blue frequencies may be a standard blue color (e.g. substantial energy around approximately 450 nm, a narrow band emission around approximately 450 nm) such that the display pixel generates standard display colors and another blue frequency may be a circadian-inducing blue (e.g., a cyan emission, substantial energy around approximately 485 nm, a narrow or broad band emission around approximately 485 nm) that is designed to effect the circadian rhythm in a more significant way by waking the person. With such a display, the display pixel colors may be changed from standard colors to represent colors accurately, according to display color standards, to display colors that are similar but not necessarily standard colors to generate an effect of the person's circadian rhythm. While the non-standard blue pixels may not be standard and may not display computer generated content in accordance with a standard color pallet, in many situations the colors may be acceptable by a user because the colors may still be acceptable while also inducing a circadian rhythm to awaken the person while using the display in the special color mode.

The CSE blue may have significant energy at a longer wavelength than the typical blue used in a display. The inventors have appreciated that longer wavelengths in the blue and cyan regions (e.g. wavelengths between the typical display blue and typical display green) may be used to both generate acceptable colors in the computer-generated content and also have a greater effect on a person's circadian rhythm. In some embodiments, the energy may be provided in a narrow band (e.g. a typical LED narrow band emission spectra with a maximum energy between 460 nm and 500 nm, 460 nm and 480 nm, 470 nm and 480 nm, or 490 nm and 500 nm). In embodiments, the energy may be more broadly spread (e.g. through the use of a phosphor or quantum dot structure) such that there is significant energy produced in the region between 460 nm and 500 nm. In such broad width systems the maximum energy may or may not fall within the 460 nm to 500 nm region. For example, the peak may be at or near the typical display blue of 450 nm and also have significant energy in the 460 nm to 500 nm region. The significant energy may be an intensity of more than 10%, 20%, 30%, 40%, or 50% of the maximum energy. That significant energy may fall within the regions of 460 and 470 nm, 470 nm and 480 nm, or 490 nm and 500 nm for example.

A computer display according to the principles of the present inventions may include a micro-LED array where the micro-LED array includes a pixel array formed of micro-LEDs including red, green and blue generating LEDs. In embodiments, the blue LED may be a circadian rhythm inducing blue LED (as described herein). If only three colors are arranged in the pixel array, the circadian-inducing blue for the pixel may not fall within the standard color gamut for the display but will generally generate acceptable colors while effecting the circadian rhythm. In embodiments, the pixel array includes two different color generating blue LEDs, one with a standard color for the display and one that may not be within the standard color gamut for display but that is adapted to affect the circadian rhythm to induce a waking effect. This arrangement would include four colors per pixel in the pixel array of the micro-LED array. In embodiments, the computer display includes only a portion of micro-LEDs with the circadian rhythm effecting blue. The micro-LED pixels may be built with different color generating LEDs, white LEDs with filters, LEDs with phosphors, etc.

In some embodiments, the CSE blue microLED may have a narrow emission characteristic where substantially all of the energy is produced over 120 nm or so and having a full width at half maximum (FWHM) of about 40 nm. In embodiments, the circadian-inducing blue microLED may have a broader emission characteristic. The broader emission may be developed by adding a phosphor to the microLED system, by using a number of narrow band emission microLEDs, etc. In embodiments, a filter may be associated with the microLED. For example, the desired blue color point may include an emission band that is broader than is achievable through a single narrow emission microLED so a phosphor or multiple narrow band LEDs may be used to broaden the emission and then a filter may be used to cut the broader emission down to the desired amount.

A standard color computer display may use a blue LED with a narrow emission characteristic. In some embodiments, the standard blue may be replaced with a broader band blue to add some cyan to the emission (i.e. slightly longer wavelength energy). This configuration may also include a filter to cut the long tail but maintain some emission in the circadian blue emission region.

Aspects of a computer display in some implementations may include an LCD backlit pixel array. Generally, an LCD backlit display has a backlight that generates a broadband of colors (e.g. white LEDs, white fluorescent) or one that generates narrow bands of color (e.g. red, green, and blue LEDs). Manufactures have typically adopted an arrangement where the backlight is a broadband white LED based system and each pixel of the LCD array is associated with a colored filter (e.g. red, green and blue) to produce the full color gamut for each pixel of the display. In some embodiments, the LCD pixel array includes filters to produce three colors per pixel based on a backlighting system that produces white light. The pixel filters filter the white light into red, green and blue. The backlight also generally produces a constant amount of light and the LCD's at each sub pixel color are changed to regulate the intensity of the color of the sub pixel (e.g. 256 steps based on a polarization setting at the sub pixel level). In embodiments, the blue filter is adapted to transmit light that is more effective at effecting the circadian rhythm (e.g. 485 nm). In embodiments, each pixel includes a fourth filter for a fourth sub pixel color. The fourth pixel uses a circadian blue pass filter such that light transmitting the sub pixel filter effects the circadian cycle in a more significant way than light passing through a standard blue filter in the pixel array. With the fourth filter configuration, the display may be set to use one and/or the other color of blue to form the blue in the pixels.

In some embodiments, the backlight produces red, green and blue in a sequence and only one LCD is used per pixel position such that the one LCD will turn on in sequence with the desired corresponding required color for the pixel. The sequential lighting system may then include a circadian-inducing blue color to affect the circadian rhythm. The sequential lighting system may further include two different colors of blue (e.g. standard blue and circadian blue) and the sequence cycles through all four colors. In embodiments, the circadian blue color may or may not be included in every cycle of the sequence. Reducing the number of cycles involved may have an effect on the perceived combined color of the pixel and of the effect of the circadian rhythm.

In some embodiments of the LCD configuration(s), the backlight may be modified to include enhanced emission at the circadian blue region. For example, a cyan LED may be included in the backlight itself such that it produces enough emission in the circadian blue region that it can generate adequate color for display and effect on the person's circadian rhythm. The backlight may include a broadband emission source or a narrow emission source for this purpose. The filter associated with the circadian blue pixels can then be adjusted to transmit the desired bandwidth of light in the region. Traditionally, the backlights used in a display do not produce much emission in this desired region so changing the lighting system to include more emission in this region may be desirable.

A computer display according to aspects of some implementations may include an OLED pixel array where the OLED array includes a pixel array formed of OLED sub pixels. The OLEDs may include red, green and blue generating OLEDs. In some embodiments, the OLEDs may produce white light and include filters to pass only the particular color desired for the sub pixel. In embodiments, the blue OLED or filter may be adapted to produce a circadian rhythm inducing blue color. If only three colors are arranged in the pixel array, the blue for the pixel may not fall within the standard color for the display but will generally generate acceptable colors while effecting the circadian rhythm. In embodiments, the pixel array may include two different color blue OLEDs, one with a standard color for the display and one that may not be within the standard color gamut for display but that is adapted to affect the circadian rhythm wake cycle. This arrangement would include four colors per pixel in the pixel array of the OLED array. In embodiments, the computer display includes only a portion of OLEDs with the circadian rhythm effecting blue.

In some embodiments, the OLED may produce a broadband of light in the region and be filtered. In embodiments, the circadian-inducing OLED may produce a narrow band emission and possibly be filtered or not.

In some implementations aspects relate to the inclusion of more than three standard colors in a computer display pixel array. The more than three colors may include the addition of a color(s) that is intended to provide a bioactive display that is switchable between a standard color gamut and a modified color gamut. The modification to the pixel colors may be adapted to produce pixel colors that can affect a person's physiological and psychological functions while maintaining the display as an effective computer display for the presentation of digital content. A control system, computer processor associated with the display may be used, either automatically (e.g., based on sensed conditions, based on time of day, based on a schedule) or through a user interface, to switch between the two modes. Such a system may also be operated in a mode where both a standard blue and circadian blue are operated simultaneously or through a rapid switching mode (e.g. pulse width modulation to regulate the apparent intensity of each one). The modified color pixel array is bioactive and may be regulated by the control system and/or a computer system to determine dose or aliquot of light of a particular characteristic or mode by a change the pixel colors over time to assist in regulating the person's circadian cycle or other physiological and psychological functions.

In some implementations the red spectrum may be positioned in the long red near infrared energy (LRNE) region. In some instances the system may switch between the two modes. Such a system may also be operated in a mode where both a standard red and LRNE red are operated simultaneously or through a rapid switching mode (e.g. pulse width modulation to regulate the apparent intensity of each one). The modified color pixel array is bioactive and may be regulated by the control system. In some instances, red in the LRNE non-visible region also referred to as near infrared may be used simultaneously or through a rapid switching mode with red or long red.

Various bioactive lighting pixel constructions may be built into a display in accordance with the principles of the present inventions. These examples are simplified examples of the basic construction of the various display technologies at a pixel level. The three examples presented are the microLED, OLED, and backlit LCD. Each of these examples uses a pixel technology that generates light at the pixel level that is outside of the normal display color gamut and at a color point or frequency that is known to affect a person's circadian rhythm and other physiological and psychological functions.

Aspects of the present invention may relate to a computer display edge lighting system or peripheral. An edge lighting system may surround the computer display and emit light that effects the circadian rhythm of a person using or proximate the computer display. The edge lighting system may include a lighting system similar to the display lighting systems described herein or a panel lighting system as described herein. The edge lighting system may be coordinated with the pixels of the display (e.g. through a computer system associated with both devices). It may otherwise be controlled separately (e.g. as described herein).

### Types of Circadian Lighting Systems for Display Systems

Lighting systems that may be used in display systems in accordance with the principles of the present inventions include, for example, 2-channel, 3-channel, 4-channel, 5-channel, or 6-channel LED-based color-tuning systems. Individual channels within the multi-channel systems may have particular color points and spectral power distributions for the light output generated by the channel. As used herein, the term “channel” refers to all the components in a light-generating pathway from an LED (microLED, OLED) through any filtering or other components until the light exits the display system.

In some implementations, 2-channel systems can be used having two white light channels. The two white light channels can be those described more fully in U.S. Provisional Patent Application No. 62/757,664, filed Nov. 8, 2018, entitled “Two Channel Tunable Lighting Systems with Controllable Equivalent Melanopic Lux and Correlated Color Temperature Outputs,” and International Patent Application No. PCT/US2019/013356, filed Jan. 11, 2019, entitled “Two-Channel Tunable Lighting Systems With Controllable Equivalent Melanopic Lux And Correlated Color Temperature Outputs” the entirety of which is incorporated herein for all purposes.

In some aspects, the present disclosure provides for display systems that incorporate two white lighting channels, which can be referred to herein as a first lighting channel and a second lighting channel. The white lighting channels can be used to backlight a display system that utilizes color filtering in order to generate a digital display.

### Types of User Interfaces and Control Systems for the Control of the Circadian Lighting

Various lighting systems and control system exemplars and aspects thereof may be implemented in accordance with the present disclosure. Although aspects of methods, systems and devices are discussed below, but it will be appreciated that the disclosure is not limited to those particular configurations, and may be applied to any combination of devices, computing systems, control systems, data centers, structures, and the like.

At a simplified level, aspects of the system and method disclosed herein include utilizing hardware referred to as computing or smart devices which may include internet streaming, desktop computers, laptops, tablets, smart phones, and sensors, to acquire, receive, measure or otherwise capture and then transmit via signal communication data associated with biological aspects of a user or data concerning the exposure of a user to variables discussed herein.

It is appreciated by those of ordinary skill in the art that some of the circuits, components, modules, and/or devices of the system disclosed in the present application are described as being in signal communication with each other, where signal communication refers to any type of communication and/or connection between the circuits, components, modules, and/or devices that allows a circuit, component, module, and/or device to pass and/or receive signals and/or information from another circuit, component, module, and/or device. The communication and/or connection may be along any signal path between the circuits, components, modules, and/or devices that allows signals and/or information to pass from one circuit, component, module, and/or device to another and includes wireless or wired signal paths. The signal paths may be physical such as, for example, conductive wires, electromagnetic wave guides, attached and/or electromagnetic or mechanically coupled terminals, semi-conductive or dielectric materials or devices, or other similar physical connections or couplings. Additionally, signal paths may be non-physical such as free-space (in the case of electromagnetic propagation) or information paths through digital components where communication information is passed from one circuit, component, module, and/or device to another in varying analog and/or digital formats without passing through a direct electromagnetic connection. These information paths may also include analog-to-digital conversions (“ADC”), digital-to-analog (“DAC”) conversions, data transformations such as, for example, fast Fourier transforms (“FFTs”), time-to-frequency conversations, frequency to-time conversions, database mapping, signal processing steps, coding, modulations, demodulations, etc.

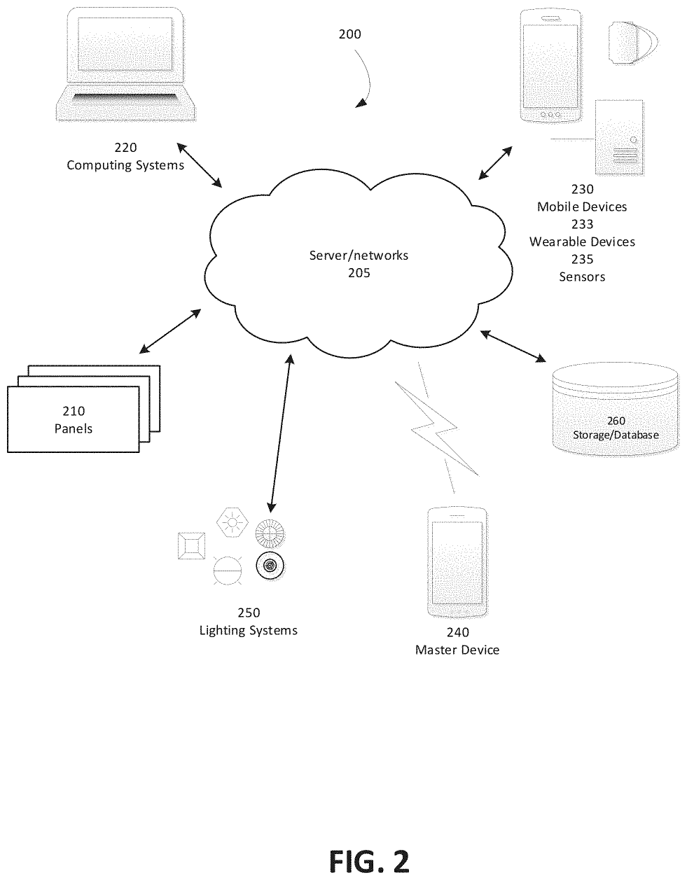

As illustrated in FIG. 2, an integrated control system may connect one or more external systems, input, and information to provide bioactive lighting, as discussed herein, through a plurality of devices, systems, and modalities. In various examples, the control system may communicate over one or more computing systems using one or more servers and networks 205 in communication with one another (e.g., network, Bluetooth, wired, wireless communication, etc.).

In some embodiments, lighting systems associated with each device may be managed by a master device 240, configured to communicate various lighting levels, timing, and configuration, for example, to achieve the desired bioactive lighting. Such levels may vary based on one or more of time of day, intended effect of the lighting, individual preferences, capabilities of the device, feedback mechanisms, sensor input, and more.

Control systems may comprise a variety of devices, including but not limited to panels and panel systems 210, computing systems 220, laptops, mobile devices 230, wearable devices 233, sensors 235, lighting systems 250 including but not limited to home, office, vehicle, and industrial lighting systems. The master device 240 may be a mobile device, computing systems, as discussed further below, and may be manually managed, automated, incorporated with machine learning, located in the cloud, and more.

In an example, lighting systems that may be used in a bioactive device including but not limited to wearable devices 233, computer display system and/or bioactive panel system 210 in accordance with the principles of the present disclosure may be controlled over time to supplement, treat or otherwise effect biological system and cycles of an exposed user throughout the day in different ways. The lighting systems may be automatically, semi-automatically or manually adjusted. The lighting systems may be adjusted based on sensor data, activity data, social media data, etc.

In some embodiments, as the panel 210 systems are installed in the environment of a lighting installation, networking features automatically engage upon powering up one or more the panel systems, and the panel systems may automatically commission themselves, such as by connecting to an overall control platform and/or to other panel systems. Thus, the panel systems in an installation may self-commission and self-configure to create a network connection between the panel systems in the environment and a remote operator (such as in the cloud). The panel systems may configure in a master/slave, ring, mesh, or peer-to-peer network, by which autonomous control features may be engaged in the environment. In embodiments, remote control features may be engaged using the network connection to the platform or other remote operators.

In some embodiments, networked communication may be used among components in the control system 200 in a deployed lighting installation that includes panel systems. Once installed and commissioned, control of the lighting installation may be handed over to an operator of a platform, such as a building owner, occupant, landlord, tenant, or the like. In embodiments, handoff may include using identity and authentication features, such as using keys, passwords, or the like that allow operation of the lighting installation by permitted users. In some embodiments, a remote-control interface of the platform may be used by an operator for remote operation of the lighting installation. The remote-control interface may use a lighting project data structure as a source of knowledge about the properties, configurations, control capabilities, and other elements of a lighting installation, so that the same platform used for the design of the lighting installation may be used to control the lighting installation. The remote-control interface may include operational guidance features, such as guiding users through the operation of a lighting installation.

In some embodiments, an autonomous control system may be provided for a lighting installation that includes panel systems of the present disclosure, by which the lighting installation may control various features of the lighting system, such as based on information collected locally in the environment, such as from one or more sensors 230. For example, the autonomous control system may automatically adjust control parameters for a light source, including but not limited to panel systems, to achieve improved adherence to the overall specifications for a lighting installation, may adjust timing variables based on detected usage patterns in a space, may adjust lighting properties based on changes in a space (such as changes in colors paints, carpet and fabrics), and the like.

Under operation, the lighting installation may include an operational feedback system, configured to collect information about the lighting installation, which may include interfaces for soliciting and receiving user feedback (such as regarding satisfaction with the installation or indicating desired changes) and which may include a sensor system 230, e.g., a lighting installation sensor system, such as including light sensors, motion sensors, temperature sensors, and others to collect information about the actual lighting conditions in the environment, activities of occupants within the environment, and the like. Information collected by the lighting installation sensor system may be relayed to a validation system of the lighting platform, such as for validation that an installation is operating as designed, including by comparison of light properties at various locations in the environment with the specifications and requirements provided in the lighting design environment, such as reflected in the lighting project data structure. In embodiments, the variances from the specifications and requirements may be provided to the autonomous control system and/or the remote-control system, so that adjustments may be made, either autonomously or by a local or remote operator of the lighting installation, to enable adjustments (such as to colors, intensities, color temperatures, beam directions, and other factors), such as to cause the lighting installation to better meet the specifications and requirements. The operational feedback system may also capture feedback that leads to revisiting the lighting design in the lighting design environment, which may induce further iteration, resulting in changes to control parameters for the panel systems, as well as automated ordering of additional or substitute panel systems, with updated installation and operational guidance.

In some embodiments, remote control may enable field programmable lighting systems, such as for transitional environments like museums (where art objects change regularly), stores (where merchandise shifts) and the like as well as for customizable environments (such as personalizing lighting in a hotel room according to a specification for a guest (which may include having the guest select an aesthetic filter) or personalized lighting for a workstation for an employee in an office setting, or personalized wearable systems. Such features may enable the lighting installation to change configurations (such as among different aesthetic filters) for multi-use environments, multi-tenant environments, and the like where lighting conditions may need to change substantially over time.

In some embodiments, a lighting system may include navigation features, such as being associated with beacons, where the lighting system interacts with one or more devices to track users within a space. The panel systems and their locations may be associated with a map, such as the map of the lighting space in the design environment. The map may be provided from the lighting design environment to one or more other location or navigation systems, such that locations of panel systems may be used as known locations or points of interest within a space.

In some embodiments, the lighting installation may be designed for an operation that is coordinated with one or more external systems, e.g., lighting, panel, and computer systems, which may serve as inputs to the lighting installation, such as music, video and other entertainment content (such as to coordinate lighting with sound). Inputs may include voice control inputs, which may include systems for assessing tone or mood from vocal patterns, such as to adjust lighting based on the same.

With respect to FIGS. 2-4 external systems can include, but are not limited to one or more computing environments, networks, local devices, remote devices, mobile devices, and wearable technology. In addition, each of those systems may provide the external input utilizable with control systems and embodiments discussed herein. For example, external inputs may include, but are not limited to audible, tactile, sensory, and user information through one or more sensors and other means, depending on the external system and its capabilities. As used herein, external systems and external information may also comprise the same types systems and information discussed below and in various embodiments herein.

In some embodiments, inputs may also include inputs from sensors associated with wearable devices 230, such as enabling adjustment of lighting control parameters (autonomously or with remote or local control features) based on physiological factors, such as ones indicating health conditions, emotional states, moods, or the like. Inputs from wearable devices may be used in the operational feedback system, such as to measure reactions to lighting conditions (such as to enable automated adjustment of a lighting installation), as well as to measure impacts on mood, health conditions, energy, wellness factors, and the like.

In some embodiments, the platform may be configured to change settings or parameters for a lighting installation (including but not limited to panel systems of the present disclosure, such as by using a custom tuning system) based on a variety of real time data, with a view to having the lighting installation, including panel systems included therein, best suit its environment in a dynamic way. In embodiments, data may be obtained that serves as an indicator of the emotional state or the stress level of an environment, and the lighting installation may respond accordingly to that state or stress level. In embodiments, data about the environment may be collected by a wearable device 233, such as a smartwatch, armband, or the like; for example, data may be collected on acceleration, location, ambient light characteristics, and heart rate, among other possibilities. In particular, underlying circadian rhythm in heart rate (CRHR) can be extracted from wearable devices. CRHR may be estimated using Bayesian uncertainty quantification. In embodiments, the data may be provided to the platform for analysis, including using machine learning, such as to observe physiological indicators of stress, mood, or the like under given lighting conditions. The analysis may enable model-based controls (such as where a given mood or state of the users in a room are linked to a set of control parameters appropriate for that state). In embodiments, machine learning may be used; for example, over time, by variation of parameters for lighting objects and fixtures (such as color, color temperature, illumination patterns, lighting distributions, and many others), a machine learning system may, using feedback on outcomes based at least in part on physiological data and other data collected by a wearable device, select and/or promotion lighting installation parameters that improve various measures of stress, mood, satisfaction, or the like. This may occur in real time under control of a machine learning system based on the current conditions of users or the environment. In embodiments, data collected at least in part by a physiological monitor or wearable device may be used as an input to processing logic on a lighting object that changes lighting levels or other parameters to accommodate the ‘emotional state’ of the users in an environment where the lighting object is located. In embodiments, there is memory that retains and manages function with no appreciable drain on the battery.

In some embodiments, inputs may include systems that take data harvested from sensors 235 in the lighting installation environment as well as sensors that reflect information about users, such as one or more of physiological sensors (including wearable devices, such as armbands, wrist bands, chest bands, glasses, clothing, and the like), sensors on various devices used by a user, ambient sensors, and the like. These may include sensing one or more of temperature, pressure, ambient lighting conditions, localized lighting conditions, lighting spectrum characteristics, humidity, UV light, sound, particles, pollutants, gases (e.g., oxygen, carbon dioxide, carbon monoxide and radon), radiation, location of objects or items, motion (e.g., speed, direction and/or acceleration). Where one or more wearable or physiological sensors are used, they may sense one or more of a person' s temperature, blood pressure, heart rate, oxygen saturation, activity type, activity level, galvanic skin response, respiratory rate, cholesterol level (including HDL, LDL and triglyceride), hormone or adrenal levels (e.g., Cortisol, thyroid, adrenaline, melatonin, and others), histamine levels, immune system characteristics, blood alcohol levels, drug content, macro and micro nutrients, mood, emotional state, alertness, sleepiness, and the like. Core body temperature (CBT) is a primary marker of circadian phase. Short of continuous hormone level monitoring for melatonin and cortisol, CBT is the closest one can get to establishing circadian phase with a single measure. Wearable devices may be used to measure CBT.

In embodiments, the platform may connect to or integrate with data sources of information about users, such as including social networks (Facebook™, Linkedin™, Twitter™, and the like, sources of medical records (23&Me™ and the like), productivity, collaboration and/or calendaring software (Google™, Outlook™, scheduling apps and the like), information about web browsing and/or shopping activity, activity on media streaming services (Netflix™ Spotify™, YouTube™, Pandora™ and the like), health record information and other sources of insight about the preferences or characteristics of users of the space of a lighting installation, including psychographic, demographic and other characteristics

In embodiments, the platform may use information from sources that indicate patterns, such as patterns involving periods of time (daily patterns, weekly patterns, seasonal patterns, and the like), patterns involving cultural factors or norms (such as indicating usage patterns or preferences in different regions), patterns relating to personality and preferences, patterns relating to social groups (such as family and work group patterns), and the like. In embodiments, the platform may make use of the data harvested from various sources noted above to make recommendations and/or to optimize (such as automatically, under computer control) the design, ordering, fulfillment, deployment and/or operation of a lighting installation, such as based on understanding or prediction of user behavior. This may include recommendation or optimization relating to achieving optimal sleep time and duration, setting optimal mealtimes, satisfying natural light exposure requirements during the day, and maintaining tolerable artificial light exposure levels (such as during night time). In embodiments, the platform may anticipate user needs and optimize the lighting installation to enhance productivity, alertness, emotional well-being, satisfaction, safety and/or sleep. In further embodiments, the platform may control one or more display systems of the present disclosure in accordance with the user needs of the environment based on this information.

In embodiments, the platform may store a space utilization data structure that indicates, over time, how people use the space of the lighting installation, such as indicating what hallways are more trafficked, and the like. This may inform understanding of a space, such as indicating what is an entry, what is a passage, what is a workspace, and the like, which may be used to suggest changes or updates to a lighting design. In embodiments, sensors may be used to collect and read where people have been in the space, such as using one or more video cameras, IR sensors, microwave sensors. LIDAR, ultrasound or the like. In embodiments, the platform may collect and read what adjustments people have made, such as task lamp activation and other activities that indicate how a lighting fixture is used by an individual in a space. By way of these examples, aggregate usage information may be used to optimize a lighting design and adjust other lighting designs. Based on these factors, a space may be dynamically adjusted, and the lighting model for an installation may be updated to reflect the actual installation.

In embodiments, control capabilities of the display systems may include dynamic configuration of control parameters, such as providing a dimming curve for a light source, including but not limited to a display system of the present disclosure, that is customized to the preferences of a designer or other user. This may include a selection from one or more modes, such as ones described elsewhere herein that have desired effects on mood or aesthetic factors, that have desired health effects, that meet the functional requirements, or the like.

Bioactive thresholds may, in some instances, benefit from prolonged exposure to at least one of one of CSE and LRNE. In some instances a melanopic flux of at least 10:1 may be suitable, in other instances the melanopic flux may be 20:1, 50:1, 100:1, or a greater ratio. It will be appreciated in light of the disclosure that most conventional systems simply adjust from a warm CCT to a cool CCT, which may only provide a 2:1 or 3:1 ratio of melanopic flux, which may not be enough to provide health benefits. In embodiments, the platform may include spectral tuning targets for display systems of the present disclosure that may optimize this ratio based on local installation environments. These targets, along with adjustments intensity of light (e.g., 4:1) may provide a higher ratio, such as a 10:1 ratio or greater, and thus provide greater melanopic flux ratios.

In a second mode and either in combination with the above mode or not, the platform may support an ability to shift the bias of light in a room. In embodiments, controlled variation of one or more display systems of the present disclosure in a lighting environment can contribute to generating a lighting bias typical of being outside.

In embodiments, various other programmable modes may be provided, such as display system settings where using different combinations of color light sources to achieve a given mixed color output may be optimized for efficacy, efficiency, color quality, health impact (e.g., circadian action), or to satisfy other requirements. In embodiments, the programmable modes may also include programmable dimming curves, color tuning curves, and the like (such as allowing various control interfaces, such as extra-low voltage (ELV) controllers or voltage-based dimmers to affect fixture colors, such as where a custom tuning curve provides a start point, an end point and a dimming and/or color tuning path in response to a level of dimming). In embodiments, programmable modes may use conventional tuning mechanisms, such as simple interpolation systems (which typically use two or three white color LEDs) are dimmable on a zero to ten-volt analog system, and have a second voltage-based input for adjusting the CCT of a fixture between warm and cool CCTs. The display systems as described herein can provide for tunable ranges of color points at various x, y coordinates on 1931 CIE chromaticity diagram. Because of the wide range of potential white or nonwhite colors produced by the display systems, they may be controlled by the platform that may specify a particular x, y coordinate on the CIE diagram. Lighting control protocols like DMX™ and Dali 2.0™ may achieve this result.

In embodiments, a programmable color curve for an LED driver may be input, such as through an interface of the platform, or through a desktop software interface, a mobile phone, a tablet app, or the like, that enables a user to define a start and stop point to a color tuning curve and to specify how it will be controlled by a secondary input, such as a voltage-based input (e.g., a 0 to 10-volt input) to the fixture. These may include pre-defined curves, as well as the ability to set start, end, and waypoints to define custom curves. For example, an exemplary color curve can have a starting point around 8000K biased above the black body curve, with the color curve crossing the black body around 2700K, and finishing around 1800K below the black body curve. Similarly, another exemplary curve could be programmed such that the start was 4000K well above the black body, with the end being 4000K well below the black body. By way of these examples, any adjustment would be in hue only, not CCT. Further examples may include a curve that never produces a white color, such as starting in the purple and finishing in orange. In any of these cases, these curves may be programmed into display systems via the interface of the platform, the desktop, mobile phone or tablet. In embodiments, the curves may be designed, saved, and then activated, such as using the secondary (supplemental) 0 to IO-volt input.

In embodiments, a three-channel warm dim mode may be used, such as that described more fully in U.S. Provisional Patent Application No. 62/712,182 filed Jul. 30, 2018, which is incorporated herein in its entirety for all purposes, for target applications where the “fully on” CCT falls between 3000K and 2500K. By way of these examples, as the fixture dims (via EL V control or in response to the O to I 0-volt input) the CCT may be gradually decreased to between 2500K and 1800K. In certain embodiments, the hue adjustment may all occur below the black body curve. Alternative embodiments may use a cyan channel as described elsewhere herein, either long-blue-pumped cyan or short blue-pumped cyan, and a red channel as described elsewhere herein, plus a 4000K white channel as described elsewhere herein to achieve a warm dimming mode that allows for adjustment both above and below the black body curve. In some embodiments of the three-channel warm dim mode, the white channel can have a color point within a 7-step MacAdam ellipse around any point on the black body locus having a correlated color temperature between about 3500K and about 6500K.

In certain embodiments, the display systems of the present disclosure can include a 4-channel color system as described elsewhere herein and in U.S. Provisional Patent Application No. 62/757,672 filed Nov. 8, 2018, and U.S. Provisional Application No. 62/712,191 filed Jul. 30, 2018, the contents of which are incorporated by reference herein in their entirety as if fully set forth herein, includes 3000K to 1800K CCT white color points within its range, a programmable mode may be included within the driver that adjusts color with the dimming percentage as well. In some aspects, this may be similar to a conventional control mode, except that the color control would not be on the secondary 0 to 10-volt channel, but may be activated through the primary 0 to 10-volt input channel or EL V controller. In embodiments, the “starting” color point may be the one when the fixture was “fully on.” In embodiments, the “ending” color point may be the one where the fixture is maximally dimmed. It is thus possible to make full range color change, such as purple to orange, which is slaved to the 0 to 10-volt or EL V dimming signal.

In embodiments, an optimized mode may be provided. With a 4-channel color system, there are many ways to create a single x-y point on the CIE diagram. In embodiments, the maximally efficient mode may typically be one that uses the colors that have x, y coordinates closest to the target x, y coordinate. But for best color quality, utilizing a fourth channel (and thereby requiring more light from the color in the opposite “corner”) may help provide a desired spectral power distribution. For the maximum melatonin suppression (for systems hoping to mimic circadian lighting), a higher cyan channel content may be required for CCTs of 3500K and above and minimizing cyan and blue content below 3500K. It will be appreciated in light of the disclosure that conventional systems either require expert users to understand the color balances necessary to achieve these effects (who then implement the color balances channel-by-channel) or are designed for maximum efficiency with color quality as a byproduct.

In embodiments, a digital power system is provided herein (including firmware-driven power conversion and LED current control) that controls a multichannel color system, such as a 4-channel color system, and allows for the inclusion of “modes” which may calculate the correct color balance between the various channels to provide optimized outputs. In embodiments, optimization may occur around one or more of efficacy, color quality, circadian effects, and other factors. Other modes are possible, such as optimizing for contrast, particular display requirements. It will be appreciated in light of the disclosure that this is not an exhaustive list but is representative of potential modes that could be engaged through an interface of the platform (or of a mobile, tablet or desktop application) where a color tuning curve may be specified, such that the curve is used to specify an interface between a controller and the Digital PSU in a display system. In embodiments, these modes may account for actual measured colors for each display system and calculate the correct balance of for the chosen modes, such as based on algorithms loaded into the Digital PSU microprocessor.

In embodiments, machine learning may be used, such as based on various feedback measures, such as relating to mood (stated by the user or measured by one or more sensors), noise levels (such as indicating successful utilization of a space based on a desired level of noise), returns on investment (such as where display systems are intended to promote retail merchandise), reported pain levels, measured health levels, performance levels of users (including fitness, wellness, and educational performance, among others), sleep levels, vitamin D levels, melatonin levels, and many others. In embodiments, the lighting installations including the display systems may be operated or controlled based on external information, such as based on seasonal lighting conditions, weather, climate, collective mood indicators (such as based on stock market data, news feeds, or sentiment indices), analyses of social network data, and the like. This may include controlling a system to reflect, or influence, the mood of occupants.

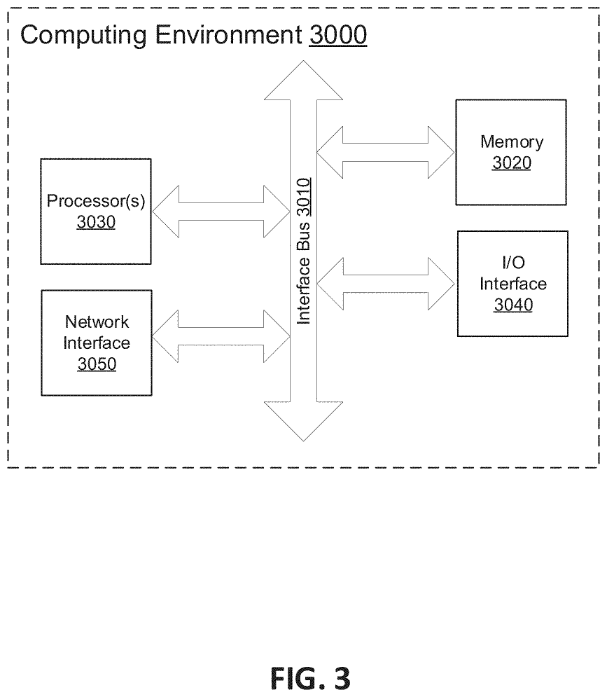

FIG. 3 depicts an example computing environment 3000 suitable for implementing aspects of the embodiments of the present invention, including the control system, which can integrate one or more devices, computing, and lighting systems. As utilized herein, the phrase “computing system” generally refers to a dedicated computing device with processing power and storage memory, which supports operating software that underlies the execution of software, applications, and computer programs thereon. As used herein, an application is a small, in storage size, specialized program that is downloaded to the computing system or device. In some cases, the application is downloaded from an “App Store.” After download, the application is generally installed on the computer system or computing device. As shown by FIG. 3, computing environment 3000 includes bus 3010 that directly or indirectly couples the following components: memory 3020, one or more processors 3030, I/0 interface 3040, and network interface 3050. Bus 3010 is configured to communicate, transmit, and transfer data, controls, and commands between the various components of computing environment 3000.

Computing environment 3000 typically includes a variety of computer readable media. Computer-readable media can be any available media that is accessible by computing environment 3000 and includes both volatile and nonvolatile media, removable and non-removable media. Computer-readable media may comprise both computer storage media and communication media. Computer storage media does not comprise, and in fact explicitly excludes, signals per se.

Computer storage media includes volatile and nonvolatile, removable and non-removable, tangible and non-transient media, implemented in any method or technology for storage of information such as computer-readable instructions, data structures, program modules or other data. Computer storage media includes RAM; ROM; EE-PROM; flash memory or other memory technology; CD-ROMs; DVDs or other optical disk storage; magnetic cassettes, magnetic tape, magnetic disk storage or other magnetic storage devices; or other mediums or computer storage devices which can be used to store the desired information and which can be accessed by computing environment 3000.

Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, communication media includes wired media, such as a wired network or direct-wired connection, and wireless media, such as acoustic, RF, infrared and other wireless media. Combinations of any of the above should also be included within the scope of computer-readable media.

Memory 3020 includes computer-storage media in the form of volatile and/or nonvolatile memory. The memory may be removable, non-removable, or a combination thereof. Memory 3020 may be implemented using hardware devices such as solid-state memory, hard drives, optical-disc drives, and the like. Computing environment 3000 also includes one or more processors 3030 that read data from various entities such as memory 3020, I/O interface 3040, and network interface 3050.

I/0 interface 3040 enables computing environment 3000 to communicate with different input devices and output devices. Examples of input devices include a keyboard, a pointing device, a touchpad, a touchscreen, a scanner, a microphone, a joystick, and the like. Examples of output devices include a display device, an audio device (e.g., speakers), a printer, and the like. These and other I/0 devices are often connected to processor 3010 through a serial port interface that is coupled to the system bus, but may be connected by other interfaces, such as a parallel port, game port, or universal serial bus (USB). A display device can also be connected to the system bus via an interface, such as a video adapter which can be part of, or connected to, a graphics processor unit. I/0 interface 3040 is configured to coordinate I/O traffic between memory 3020, the one or more processors 3030, network interface 3050, and any combination of input devices and/or output devices.

Network interface 3050 enables computing environment 3000 to exchange data with other computing devices via any suitable network. In a networked environment, program modules depicted relative to computing environment 3000, or portions thereof, may be stored in a remote memory storage device accessible via network interface 3050. It will be appreciated that the network connections shown are exemplary and other means of establishing a communications link between the computers may be used.

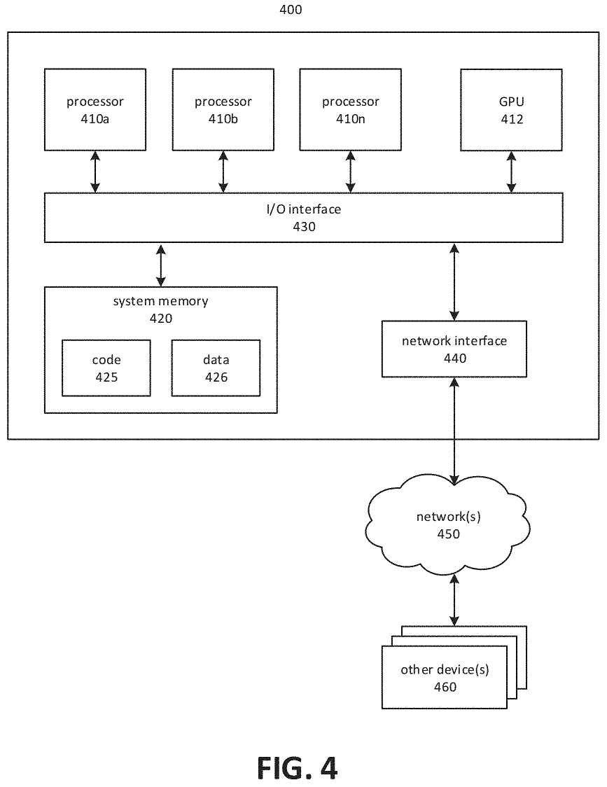

In at least some embodiments, a server that implements a portion or all of one or more of the technologies described herein may include a general-purpose computer system that includes or is configured to access one or more computer accessible media. FIG. 4 depicts a general-purpose computer system that includes or is configured to access one or more computer-accessible media. In the illustrated embodiment, computing device 400 includes one or more processors 410a, 410b, and/or 41 On (which may be referred herein singularly as a processor or in the plural as the processors 410) coupled to a system memory 420 via an input/output (“I/0”) interface 430. Computing device 400 further includes a network interface 440 coupled to I/0 interface 430.

In various embodiments, computing device 400 may be a uniprocessor system including one processor 410 or a multiprocessor system including several processors 410 (e.g., two, four, eight, or another suitable number). Processors 410 may be any suitable processors capable of executing instructions. For example, in various embodiments, processors 410 may be general-purpose or embedded processors implementing any of a variety of instruction set architectures (“ISAs”), such as the x86, PowerPC, SP ARC or MIPS ISAs, or any other suitable ISA In multiprocessor systems, each of processors 410 may commonly, but not necessarily, implement the same ISA.

In some embodiments, a graphics processing unit (“GPU”) 412 may participate in providing graphics rendering and/or physics processing capabilities. A GPU may, for example, comprise a highly parallelized processor architecture specialized for graphical computations. In some embodiments, processors 410 and GPU 412 may be implemented as one or more of the same type of device.

System memory 420 may be configured to store instructions and data accessible by processor(s) 410. In various embodiments, system memory 420 may be implemented using any suitable memory technology, such as static random access memory (“SRAM”), synchronous dynamic RAM (“SDRAM”), nonvolatile/Flash®-type memory, or any other type of memory. In the illustrated embodiment, program instructions and data implementing one or more desired functions, such as those methods, techniques, and data described above, are shown stored within system memory 420 as code 425 and data 426.

In one embodiment, I/O interface 430 may be configured to coordinate I/0 traffic between processor 410, system memory 420, and any peripherals in the device, including network interface 440 or other peripheral interfaces. In some embodiments, I/O interface 430 may perform any necessary protocol, timing or other data transformations to convert data signals from one component (e.g., system memory 420) into a format suitable for use by another component (e.g., processor 410). In some embodiments, I/O interface 430 may include support for devices attached through various types of peripheral buses, such as a variant of the Peripheral Component Interconnect (“PCI”) bus standard or the Universal Serial Bus (“USB”) standard, for example. In some embodiments, the function of I/O interface 430 may be split into two or more separate components, such as a north bridge and a south bridge, for example. Also, in some embodiments some or all of the functionality of I/O interface 430, such as an interface to system memory 420, may be incorporated directly into processor 410.

Network interface 440 may be configured to allow data to be exchanged between computing device 400 and other device or devices 460 attached to a network or networks 450, such as other computer systems or devices, for example. In various embodiments, network interface 440 may support communication via any suitable wired or wireless general data networks, such as types of Ethernet networks, for example. Additionally, network interface 440 may support communication via telecommunications/telephony networks, such as analog voice networks or digital fiber communications networks, via storage area networks, such as Fibre Channel SANs (storage area networks), or via any other suitable type of network and/or protocol.

In some embodiments, system memory 420 may be one embodiment of a computer-accessible medium configured to store program instructions and data as described above for implementing embodiments of the corresponding methods and apparatus. However, in other embodiments, program instructions and/or data may be received, sent, or stored upon different types of computer-accessible media. Generally speaking, a computer-accessible medium may include non-transitory storage media or memory media, such as magnetic or optical media, e.g., disk or DVD/CD coupled to computing device 400 via I/O interface 430. A non-transitory computer-accessible storage medium may also include any volatile or non-volatile media, such as RAM (e.g., SDRAM, DDR SDRAM, RDRAM, SRAM, etc.), ROM, etc., that may be included in some embodiments of computing device 400 as system memory 420 or another type of memory. Further, a computer-accessible medium may include transmission media or signals, such as electrical, electromagnetic or digital signals, conveyed via a communication medium, such as a network and/or a wireless link, such as those that may be implemented via network interface 440. Portions or all of multiple computing devices, such as those illustrated in FIG. 4, may be used to implement the described functionality in various embodiments; for example, software components running on a variety of different devices and servers may collaborate to provide the functionality. In some embodiments, portions of the described functionality may be implemented using storage devices, network devices or special-purpose computer systems, in addition to or instead of being implemented using general-purpose computer systems. The term “computing device,” as used herein, refers to at least all these types of devices and is not limited to these types of devices.

A compute node, which may be referred to also as a computing node, may be implemented on a wide variety of computing environments, such as tablet computers, personal computers, smartphones, game consoles, commodity-hardware computers, virtual machines, web services, computing clusters, and computing appliances. Any of these computing devices or environments may, for convenience, be described as compute nodes or as computing nodes.

A network set up by an entity, such as a company or a public sector organization, to provide one or more web services (such as various types of cloud-based computing or storage) accessible via the Internet and/or other networks to a distributed set of clients may be termed a provider network. Such a provider network may include numerous data centers hosting various resource pools, such as collections of physical and/or virtualized computer servers, storage devices, networking equipment, and the like, needed to implement and distribute the infrastructure and web services offered by the provider network. The resources may in some embodiments be offered to clients in various units related to the web service, such as an amount of storage capacity for storage, processing capability for processing, as instances, as sets of related services, and the like. A virtual computing instance may, for example, comprise one or more servers with a specified computational capacity (which may be specified by indicating the type and number of CPUs, the main memory size, and so on) and a specified software stack (e.g., a particular version of an operating system, which may in turn run on top of a hypervisor).

A number of different types of computing devices may be used singly or in combination to implement the resources of the provider network in different embodiments, including general-purpose or special-purpose computer servers, storage devices, network devices, and the like. In some embodiments a client or user may be provided direct access to a resource instance, e.g., by giving a user an administrator login and password. In other embodiments the provider network operator may allow clients to specify execution requirements for specified client applications and schedule execution of the applications on behalf of the client on execution platforms (such as application server instances, Java™ virtual machines (“JVMs”), general-purpose or special-purpose operating systems, platforms that support various interpreted or compiled programming languages, such as Ruby, Perl, Python, C, C++, and the like, or high-performance computing platforms) suitable for the applications, without, for example, requiring the client to access an instance or an execution platform directly. A given execution platform may utilize one or more resource instances in some implementations; in other implementations multiple execution platforms may be mapped to a single resource instance.

In many environments, operators of provider networks that implement different types of virtualized computing, storage and/or other network-accessible functionality may allow customers to reserve or purchase access to resources in various resource acquisition modes. The computing resource provider may provide facilities for customers to select and launch the desired computing resources, deploy application components to the computing resources, and maintain an application executing in the environment. In addition, the computing resource provider may provide further facilities for the customer to quickly and easily scale up or scale down the numbers and types of resources allocated to the application, either manually or through automatic scaling, as demand for or capacity requirements of the application change. The computing resources provided by the computing resource provider may be made available in discrete units, which may be referred to as instances. An instance may represent a physical server hardware platform, a virtual machine instance executing on a server, or some combination of the two. Various types and configurations of instances may be made available, including different sizes of resources executing different operating systems (“OS”) and/or hypervisors, and with various installed software applications, runtimes, and the like. Instances may further be available in specific availability zones, representing a logical region, a fault tolerant region, a data center, or other geographic location of the underlying computing hardware, for example. Instances may be copied within an availability zone or across availability zones to improve the redundancy of the instance, and instances may be migrated within a particular availability zone or across availability zones. As one example, the latency for client communications with a particular server in an availability zone may be less than the latency for client communications with a different server. As such, an instance may be migrated from the higher latency server to the lower latency server to improve the overall client experience.

In some embodiments the provider network may be organized into a plurality of geographical regions, and each region may include one or more availability zones. An availability zone (which may also be referred to as an availability container) in turn may comprise one or more distinct locations or data centers, configured in such a way that the resources in a given availability zone may be isolated or insulated from failures in other availability zones. That is, a failure in one availability zone may not be expected to result in a failure in any other availability zone. Thus, the availability profile of a resource instance is intended to be independent of the availability profile of a resource instance in a different availability zone. Clients may be able to protect their applications from failures at a single location by launching multiple application instances in respective availability zones. At the same time, in some implementations inexpensive and low latency network connectivity may be provided between resource instances that reside within the same geographical region (and network transmissions between resources of the same availability zone may be even faster).

Each of the processes, methods, and algorithms described in the preceding sections may be embodied in, and fully or partially automated by, code modules executed by one or more computers or computer processors. The code modules may be stored on any type of non-transitory computer-readable medium or computer storage device, such as hard drives, solid state memory, optical disc, and/or the like. The processes and algorithms may be implemented partially or wholly in application-specific circuitry. The results of the disclosed processes and process steps may be stored, persistently or otherwise, in any type of non-transitory computer storage, such as, e.g., volatile or nonvolatile storage.

The various features and processes described above may be used independently of one another, or may be combined in various ways. All possible combinations and sub-combinations are intended to fall within the scope of this disclosure. In addition, certain methods or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

It will also be appreciated that various items are illustrated as being stored in memory or on storage while being used, and that these items or portions thereof may be transferred between memory and other storage devices for purposes of memory management and data integrity. Alternatively, in other embodiments some or all of the software modules and/or systems may execute in memory on another device and communicate with the illustrated computing systems via inter-computer communication. Furthermore, in some embodiments, some or all of the systems and/or modules may be implemented or provided in other ways, such as at least partially in firmware and/or hardware, including, but not limited to, one or more application-specific integrated circuits (“ASICs”), standard integrated circuits, controllers (e.g., by executing appropriate instructions, and including microcontrollers and/or embedded controllers), field programmable gate arrays (“FPGAs”), complex programmable logic devices (“CPLDs”), etc. Some or all of the modules, systems, and data structures may also be stored (e.g., as software instructions or structured data) on a computer-readable medium, such as a hard disk, a memory, a network, or a portable media article to be read by an appropriate device or via an appropriate connection. The systems, modules, and data structures may also be transmitted as generated data signals (e.g., as part of a carrier wave or other analog or digital propagated signal) on a variety of computer-readable transmission media, including wireless-based and wired/cable-based media, and may take a variety of forms (e.g., as part of a single or multiplexed analog signal, or as multiple discrete digital packets or frames). Such computer program products may also take other forms in other embodiments. Accordingly, the present invention may be practiced with other computer system configurations.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

While certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions and changes in the form of the methods and systems described herein may be made without departing from the spirit of the inventions disclosed herein. The accompanying claims and their equivalents are intended to cover such forms or modifications as would fall within the scope and spirit of certain of the inventions disclosed herein.

What is claimed:

1. A computer-implemented method, comprising:
obtaining information about a photic environment, the information including at least one light metric;
tracking the at least one metric over a period of time;
generating a dosage level based on information about the photic environment, the tracked metric, and an intended circadian response, wherein the dosage level includes a light level; and
outputting the dosage level on at least one light-emitting device.

2. The method of claim 1, wherein the at least one light metric of the photic environment comprises at least one of a daytime light intensity, a morning light intensity, an afternoon light intensity, an evening light intensity, a nighttime light intensity, a duration of night and day, a timing of dawn and dusk, and a fraction of time spent in light intensity zone.

3. The method of claim 1, wherein the at least one light metric of the photic environment comprises at least one of a photopic lux, a melanopic lux, a circadian potency, a circadian light, and a scotopic lux.

4. The method of claim 1, wherein obtaining information about the photic environment comprises inferring, from non-light information, including at least one of a wearable device, a time, and a location.

5. The method of claim 1, further comprising processing the information using a dose response curve.

6. The method of claim 5, wherein the dose response curve is based on a light level.

7. The method of claim 1, wherein the dosage level comprises an exposure time.

8. The method of claim 7, wherein the light level of the dosage level varies based on at least one of a time and time of day.

9. The method of claim 1, wherein generating the dosage level utilizes a phase angle between two or more zeitgebers.

10. The method of claim 1, the generated dosage level is further based, at least in part, on at least one of an equivalent latitude, an equivalent longitude, and a risk factor.

11. The method of claim 1, the wherein the intended circadian response is one or more of improved sleep, increased immunity, support of weight loss, improved mental health, improved fertility, improved athletic performance, and improved academic performance.

12. A system, comprising:
at least one light-emitting device;
at least one processor in communication with the at least one light-emitting device; and
a memory in communication with the at least one processor, the memory comprising instructions executable by the at least one processor to at least:
obtain information about a photic environment, the information including at least one light metric;
track the at least one metric over a period of time;
generate a dosage level based on information about the photic environment, the tracked metric, and an intended circadian response, wherein the dosage level includes a light level; and
output the dosage level on at least one light-emitting device.

13. The system of claim 12, wherein the at least one processor and memory are in a master device, which may be local or remote to the photic environment.

14. The system of claim 12, wherein the at least one light-emitting device is one of a computer display, a LCD configuration, an OLED array, a mobile device, a television display, a household, a SAD lamp, a lamp, a monitor, a blood pressure monitor, a wall panel, a light fixture, and a multi-channel display system.

15. The system of claim 12, wherein the at least one light metric of the photic environment comprises at least one of a daytime light intensity, a morning light intensity, an afternoon light intensity, an evening light intensity, a nighttime light intensity, a duration of night and day, a timing of dawn and dusk, and a fraction of time spent in light intensity zone.

16. The system of claim 12, wherein the at least one light metric of the photic environment comprises at least one of a photopic lux, a melanopic lux, a circadian potency, a circadian light, and a scotopic lux.

17. The system of claim 12, wherein obtaining information about the photic environment comprises inferring, from non-light information, including at least one of a wearable device, a time, and a location.

18. The system of claim 12, comprising a plurality of light-emitting devices, and wherein the memory further comprises instructions executable by the at least one processor to output different dosages on the plurality of light-emitting devices.

19. The system of claim 12, wherein the memory further comprises instructions executable by the at least one processor to:
modify the dosage level on the at least one light-emitting device based on feedback from the photic environment.

20. A non-transitory computer-readable medium comprising instructions executable by at least one processor to perform a method, the method comprising:
obtaining information about a photic environment, the information including at least one light metric;
tracking the at least one metric over a period of time;
generating a dosage level based on information about the photic environment, the tracked metric, and an intended circadian response, wherein the dosage level includes a light level; and
outputting the dosage level on at least one light-emitting device.
